(19) United States
(12) Patent Application Publication
(10) Pub. No.: US 2008/0147007 A1
(43) Pub. Date: Jun. 19, 2008

(54) DELIVERY DEVICE WITH PRESSURE CONTROL

(76) Inventors: Toby FREYMAN, Waltham, MA (US); Timothy J. Mickley, Corcoran, MN (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 11/612,542

(22) Filed: Dec. 19, 2006

(51) Int. Cl. A61M 1/00; B65D 71/00

(52) U.S. Cl. 604/151; 206/223

(57) ABSTRACT

Delivery devices with pressure control mechanisms are described herein. In some embodiments an apparatus includes a reservoir configured to contain a fluid, the reservoir having an inlet port and an outlet port. A plunger is disposed within the reservoir such that the reservoir is divided into a first portion and a second portion. The inlet port is in fluid communication with the first portion of the reservoir, and the outlet port is in fluid communication with the second portion of the reservoir. A pressure control device configured to control a pressure of the fluid within the first portion of the reservoir is in fluid communication with the first portion of the reservoir.

### BACKGROUND

The invention relates generally to medical devices, and more particularly to medical devices configured to deliver therapeutic fluids into the body at a controlled pressure.

Medical techniques, such as those involving regenerative therapy, require the direct introduction of therapeutic materials, such as drugs, proteins, molecules and/or living cells into the body. For example, some known techniques for repairing myocardial tissue require the introduction of living cells into the affected region, either by directly injecting the cells into the affected tissue or by injecting the cells into the vasculature adjacent the affected tissue. The introduction of cells is generally accomplished using a cell delivery catheter.

Studies have shown that the methods and conditions under which cells are introduced into the body can impact the survival of the cells and/or the effectiveness of the cellular therapy. More specifically, studies have shown that limiting the shear stress and controlling the delivery pressure and/or flow rate of the cells can benefit the effectiveness of the therapy. Some known cell delivery catheters use a manually actuated piston or plunger to generate the pressure, and force the cells, from a reservoir through the catheter to the targeted area. Such catheters often do not include a mechanism for controlling the pressure of the cell solution, but rather rely solely on the user to control the rate at which the plunger is displaced and/or the pressure that the plunger applies to the cell solution. Other known cell delivery catheters use complex and expensive external mechanisms, such as stepper motors, to control the rate at which the plunger is displaced. Yet other known cell delivery catheters control the delivery pressure using valves in fluid communication with the cell solution that, when actuated, result in the cells being exposed to high shear stress.

Thus, there is a need for a cell delivery device that controls the delivery pressure of the cell solution in a non-complex manner without causing damage to the cells.

### SUMMARY

Delivery devices with pressure control devices are described herein. In some embodiments an apparatus includes a reservoir configured to contain a fluid, the reservoir having an inlet port and an outlet port. A movable member is disposed within the reservoir such that the reservoir is divided into a first portion and a second portion. The inlet port is in fluid communication with the first portion of the reservoir, and the outlet port is in fluid communication with the second portion of the reservoir. A pressure control device configured to control a pressure of the fluid within the first portion of the reservoir is in fluid communication with the first portion of the reservoir.

### BRIEF DESCRIPTION OF THE DRAWINGS

FIGS. 1 and 2 are schematic illustrations of a delivery device according to an embodiment of the invention in a first position and a second position, respectively.

FIG. 3 is a partial cross-sectional view of a delivery device according to an embodiment of the invention.

FIGS. 4 and 5 are cross-sectional views of the area labeled 4 of the delivery device illustrated in FIG. 3 in a closed position and a bypass position, respectively.

FIG. 6 is a schematic illustration of the plunger of the delivery device illustrated in FIG. 3.

FIG. 7 is cross-sectional view of a pressure control device according to an embodiment of the invention.

FIG. 8 is a partial cross-sectional view of a delivery device having a first and a second pressure control device according to an embodiment of the invention.

FIG. 9 is a cross-sectional view of the area labeled 9 of the delivery device illustrated in FIG. 8.

FIG. 10 is a cross-sectional view of portion of a delivery device according to an embodiment of the invention.

FIG. 11 is a cross-sectional view of portion of a delivery device having a biasing member according to an embodiment of the invention.

FIGS. 12 and 13 are schematic illustrations of a delivery device according to an embodiment of the invention in a first position and a second position, respectively.

FIG. 14 is a partial cross-sectional view of a second reservoir according to an embodiment of the invention.

FIG. 15 is a partial cross-sectional view of a first reservoir according to an embodiment of the invention.

FIG. 16 is a partial cross-sectional view of a delivery device according to an embodiment of the invention that includes the second reservoir illustrated in FIG. 14 and the first reservoir illustrated in FIG. 15.

FIGS. 17 and 18 are schematic illustrations of a delivery device according to an embodiment of the invention in a first configuration and a second configuration, respectively.

### DETAILED DESCRIPTION

Delivery devices with pressure control devices are described herein. In some embodiments an apparatus includes a reservoir configured to contain a fluid, the reservoir having an inlet port and an outlet port. A movable member is disposed within the reservoir such that the reservoir is divided into a first portion and a second portion. The inlet port is in fluid communication with the first portion of the reservoir, and the outlet port is in fluid communication with the second portion of the reservoir. A pressure control device, such as a check valve, configured to control a pressure of the fluid within the first portion of the reservoir is in fluid communication with the first portion of the reservoir. In some embodiments, the apparatus includes a drain tube coupled to the pressure control device, the drain tube being configured to provide an indication that the fluid is passing therethrough.

In other embodiments, an apparatus includes a reservoir configured to contain a fluid, the reservoir having an inlet port and an outlet port. A movable member, such as, for example, a plunger is disposed within the reservoir such that the reservoir is divided into a first portion and a second portion. The movable member has a first portion in fluid communication with the first portion of the reservoir and a second portion in fluid communication with the second portion of the reservoir, the first portion of the movable member having a first area and the second portion of the movable member having a second area, the second area being different from the first area. The inlet port is in fluid communication with the first portion of the reservoir, and the outlet port is in fluid communication with the second portion of the reservoir. A pressure control device configured to control a pressure of the fluid within the first portion of the reservoir is in fluid communication with the first portion of the reservoir.

In other embodiments, an apparatus includes a first reservoir configured to contain a first fluid, a second reservoir configured to retain a second fluid, a movable member and a valve. The first reservoir includes a port, such as, for example a port configured to receive the first fluid from an external fluid supply source. The second reservoir includes a port, such as, for example, a port including a quick-connect fitting configured to couple the second reservoir to a supply catheter. The movable member has a first end portion configured to be disposed within the first reservoir and a second end portion configured to be disposed within the second reservoir. The valve is coupled to the first reservoir and is configured to control a pressure of the first fluid within the first reservoir.

In yet other embodiments, an apparatus includes a reservoir configured to contain a fluid, an expandable member and a pressure control device. The expandable member is disposed within the reservoir such that the reservoir is divided into a first portion and a second portion. The reservoir includes an inlet port, which is in fluid communication with the first portion, and an outlet port, which is in fluid communication with the second portion. The pressure control device is in fluid communication with the first portion of the reservoir and is configured to control a pressure of the fluid within the first portion of the reservoir.

In yet other embodiments, a kit includes a reservoir configured to contain a fluid, a movable member, a pressure control device and a fluid supply member. The movable member is disposed within the reservoir such that the reservoir is divided into a first portion and a second portion. The reservoir includes an inlet port, which is in fluid communication with the first portion, and an outlet port, which is in fluid communication with the second portion. The pressure control device, such as, for example, a check valve, is in fluid communication with the first portion of the reservoir and is configured to control a pressure of a fluid within the first portion of the reservoir. The fluid supply member is configured to supply the fluid to the first portion of the reservoir. The fluid supply member can be, for example, a syringe.

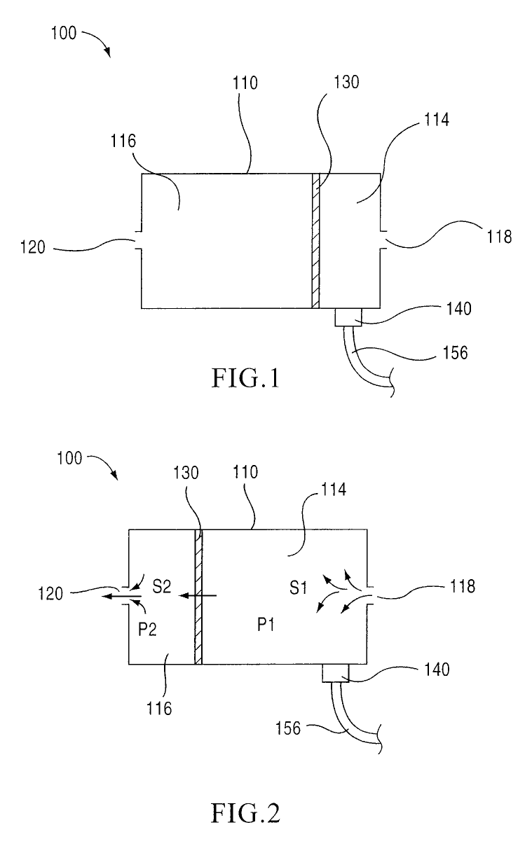

FIGS. 1 and 2 are schematic illustrations of a delivery device 100 according to an embodiment of the invention in a first position and a second position, respectively. The illustrated delivery device 100 includes a reservoir 110 configured to contain a fluid and a movable member 130 disposed within the reservoir 110. The movable member 130, which can be, for example, a piston or a plunger, is disposed within the reservoir 110 such that the reservoir 110 is divided into a first portion 114 and a second portion 116 that is fluidically isolated from the first portion 114. The reservoir 110 includes a first port 118 in fluid communication with the first portion 114 and a second port 120 in fluid communication with the second portion 116. As discussed in more detail herein, the first port 118 can be, for example, an inlet port configured to be connected to a fluid supply source, such as a syringe (not shown). The second port 120 can be, for example, be a quick connect fitting configured to allow the second port 120 to be removably connected to a catheter tube (not shown) configured to convey a fluid from the second portion 116 of the reservoir 110 to a target location within a patient's body. The illustrated delivery device 100 also includes a pressure control device 140 in fluid communication with the first portion 114 of the reservoir 110. The pressure control device 140 is configured to control a pressure P1 of a first fluid S1 within the first portion 114 of the reservoir 110.

In use, the second portion 116 of the reservoir 110 is filled with a predetermined amount of a second fluid S2. The second fluid S2 can be a therapeutic solution, such as for example, a solution containing living cells. In the illustrated embodiment, the second portion 116 of the reservoir 110 can be filled via the second port 120. The introduction of the second fluid S2 into the second portion 116 can cause the movable member 130 to be displaced such that the volume of the second portion 116 of the reservoir 110 is increased to accommodate the desired amount of the second fluid S2. Once filled with the desired amount of the second fluid S2, the second port 120 can then be placed in fluid communication with any suitable device (not shown), such as a tube, a catheter, or the like, configured to deliver the second fluid S2 to a target location within a patient's body. In some embodiments, the second port 120 can include a quick connect fitting, a Luer connector, or the like.

The second fluid S2 can be conveyed from the second portion 116 of the reservoir 110 by introducing a first fluid S1 into the first portion 114 of the reservoir 110. In some embodiments, the first fluid S1 can be introduced into the first portion 114 by a syringe (not shown) in fluid communication with the first port 118. The introduction of the first fluid S1 into the first portion 114 of the reservoir 110 causes the movable member 130 to move, as indicated by the arrow in FIG. 2, such that the volume of the first portion 114 increases and the volume of the second portion 116 decreases, thereby causing the second fluid S2 to be expelled from the from the second portion 116 of the reservoir 110. Put another way, the first fluid S1 acts to hydraulically or pneumatically actuate the movable member 130, thereby pushing the second fluid S2 out of the second portion 116 of the reservoir. The first fluid S1 can be a liquid or a gas. In some embodiments, for example, the first fluid can be a saline solution.

The pressure and/or rate at which the second fluid S2 is delivered from the second portion 116 of the reservoir 110 is dependent on the pressure exerted by the first fluid S1 on the movable member 130. As will be discussed in more detail herein, in some embodiments, the pressure P2 of the second fluid S2 in the second portion 116 can be proportional and/or equal to the pressure P1 of the first fluid S1 in the first portion 114. As such, the delivery pressure P2 and/or the delivery flow rate of the second fluid S2 can be controlled by controlling the pressure P1 of the first fluid S1 in the first portion 114 of the reservoir 110.

The pressure P1 of the first fluid S1 within the first portion 114 of the reservoir 110 is controlled by the pressure control device 140. The pressure control device 140 can be any device suitable for controlling fluid pressure, such as, for example, a check valve, a reed valve, a poppet valve, a diaphragm, a needle valve or any other suitable bypass valve. In some embodiments, the pressure P1 can be controlled by permitting a portion of the first fluid S1 to be released from the first portion 114 of the reservoir 110. As such, the delivery device 100 can include a drain tube 156 configured to convey the discharged portion of the first fluid S1 from the pressure control device 140 to a secondary reservoir (not shown).

Although the second fluid S2 is described as being a solution containing living cells, in some embodiments, the second fluid S2 can include any type of therapeutic material, such as drugs, genetic materials, and biological materials. Suitable genetic materials can include, for example, DNA or RNA, such as DNA/RNA encoding a useful protein and DNA/RNA intended to be inserted into a human body including viral vectors and non-viral vectors. Suitable viral vectors can include, for example, adenoviruses, gutted adenoviruses, adeno-associated viruses, retroviruses, alpha viruses (Semliki Forest, Sindbis, etc.), lentiviruses, herpes simplex viruses, ex vivo modified cells (e.g., stem cells, fibroblasts, myoblasts, satellite cells, pericytes, cardiomyocytes, skeletal myocytes, macrophage), replication competent viruses (e.g., ONYX-015), and hybrid vectors. Suitable non-viral vectors include, for example, artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD).

Suitable biological materials can include, for example, cells, yeasts, bacteria, proteins, peptides, cytokines, hormones, matrices (such as extracellular matrices), and natural polymers (such as hyaluronic acid). Examples of suitable peptides and proteins include growth factors (e.g., FGF, FGF-1, FGF-2, VEGF, Endotherial Mitogenic Growth Factors, and epidermal growth factors, transforming growth factor α and β, platelet derived endothelial growth factor, platelet derived growth factor, stem cell factor, tumor necrosis factor α, hepatocyte growth factor and insulin like growth factor), transcription factors, proteinkinases, CD inhibitors, thymidine kinase, and bone morphogenic proteins (BMP's), such as BMP-2, BMP-3, BMP-4, BMP-5, BMP-6 (Vgr-1), BMP-7 (OP-1), BMP-8. BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, and BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at a desired site. The delivery media can be formulated as needed to maintain cell function and viability. For example, the delivery media may include polymers or protein carriers for therapeutics so that the polymer increases viscosity and retention of the therapeutic material and may increase cell survival once delivered to the tissue. Cells include, for example, whole bone marrow, bone marrow derived mono-nuclear cells (BM-MNC), progenitor cells (e.g., endothelial progentitor cells (EPC)), stem cells (e.g., mesenchymal (MSC, including MSC+5-aza), hematopoietic, neuronal, cardiac, or other tissue derived, embryonic stem cells and stem cell clones), pluripotent stem cells, fibroblasts, MyoD scar fibroplasts, macrophage, side populations (SP) cells, lineage negative (Lin⁻) cells (including Lin⁻CD34⁻, Lin⁻CD34⁺, and Lin⁻cKit⁺)), cord blood cells, skeletal myoblasts, muscle-derived cells (MDC), Go cells, endothelial cells, adult myocardiomyocytes, smooth muscle cells, adult cardiac fibroplasts+5-aza, pacing cells, fetal or neonatal cells, immunologically masked cells, genetically modified cells, teratoma derived cells, and satellite cells, and tissue engineered grafts.

Therapeutic materials can also include non-genetic agents, such as, for example anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid, amlodipine and doxazosin; anti-inflammatory agents such as glucocorticoids, betamethasone, dexamethasone, prednisolone, corticosterone, budesonide, estrogen, sulfasalazine, and mesalamine; antineoplastic/antiproliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, taxol and its analogs or derivatives; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick antiplatelet peptides; vascular cell growth promotors such as growth factors, Vascular Endothelial Growth Factors (VEGF, all types including VEGF-2), growth factor receptors, transcriptional activators, and translational promotors; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents, vasodilating agents, and agents which interfere with endogenous vasoactive mechanisms; anti-oxidants, such as probucol; antibiotic agents, such as penicillin, cefoxitin, oxacillin, tobranycin; angiogenic substances, such as acidic and basic fibroblast growth factors, estrogen including estradiol (E2), estriol (E3) and 17-Beta Estradiol; and drugs for heart failure, such as digoxin, beta-blockers, angiotensin-converting enzyme (ACE) inhibitors including captopril and enalopril.

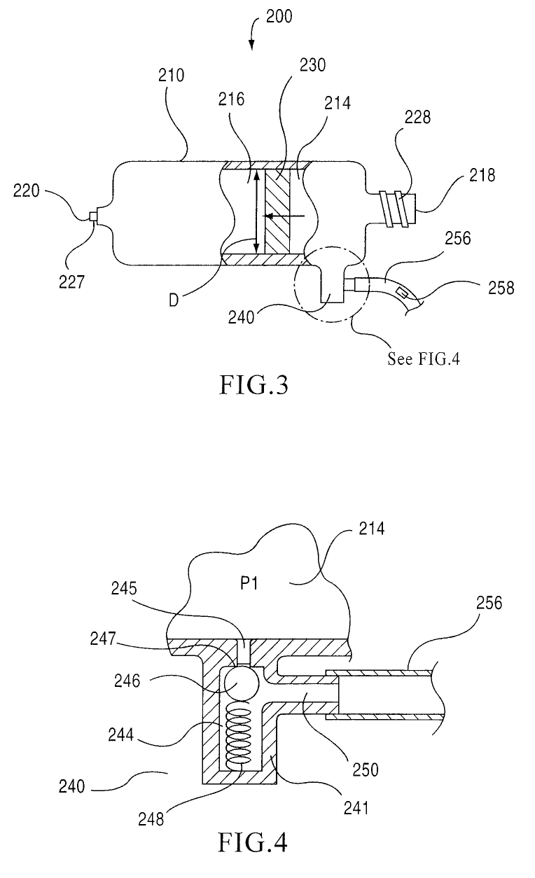

FIGS. 3-5 illustrate a delivery device 200 according to an embodiment of the invention. The illustrated delivery device 200 includes a reservoir 210 configured to contain a fluid and a plunger 230 disposed within the reservoir 210. The plunger 230 is disposed within the reservoir 210 such that the reservoir 210 is divided into a first portion 214 and a second portion 216 that is fluidically isolated from the first portion 214. In the illustrated embodiment, the reservoir 210 is a cylinder having a constant inner diameter D. The reservoir is constructed from a rigid material such that the total volume of the reservoir 210, which is the sum of the volume of the first portion 214 and the second portion 216, remains substantially constant regardless of the pressure of the fluids contained therein. Put another way, because the reservoir 210 is a rigid cylinder having a constant inner diameter D, as the plunger 230 moves, the change in the volume of the first portion 214 is equal to the change in the volume of the second portion 216.

The reservoir includes a first port 218 in fluid communication with the first portion 214 and a second port 220 in fluid communication with the second portion 216. The first port 218 can be, for example, an inlet port configured to be connected to a fluid supply source, such as a syringe (not shown). The second port 220 can be, for example, an outlet port having a quick-connect fitting, such as a Luer fitting, configured to be removably connected to a catheter tube or other device for transporting a fluid into and out of the second portion 216 of the reservoir 210. The illustrated delivery device 200 includes a valve 240 in fluid communication with the first portion 214 of the reservoir 210. A drain tube 256 having a flow indicator 258 is coupled to the valve 240. As described above, the drain tube 256 is configured to convey the fluid discharged from the first portion 214 of the reservoir 210 to a secondary reservoir. The flow indicator 258 can be a qualitative indicator, such as, for example, a transparent portion of the drain tube 256 configured to produce a qualitative indication when fluid is flowing through the drain tube 256. In other embodiments, the flow indicator 258 can be a flow measurement device configured to provide a quantitative indicator of the amount of fluid flowing through the drain tube 256.

As described above, the second portion 216 of the reservoir 210 can be filled with a predetermined amount of a therapeutic fluid, such as, for example a solution containing living cells. Once filled with the desired amount of the therapeutic fluid, the second port 220 can then be placed in fluid communication with any suitable device, such as a delivery catheter (not shown), configured to deliver the therapeutic fluid to a target location. Similarly, the first port 218 can be placed in fluid communication with a source of pressurized working fluid, such as a syringe. The therapeutic fluid can be delivered to the target location by introducing the working fluid into the first portion 214 of the reservoir, which in turn causes the plunger 230 to move as indicated by the arrow in FIG. 3, thereby pushing the therapeutic fluid out of the second portion 216 of the reservoir 210.

The pressure and/or rate at which the therapeutic fluid is delivered from the second portion 216 of the reservoir 210 is a function of the pressure exerted by the working fluid on the plunger 230. FIG. 6 is a schematic illustration showing the forces acting on the plunger 230 when the delivery device 200 is in use. The forces include pressure forces F1 and F2 caused by the pressure P1 of the working fluid in the first portion 214 and the pressure P2 of the therapeutic fluid in the second portion 216, respectively. As illustrated, forces F1 and F2 act in a direction normal to the surfaces of the ends of the plunger 230 (i.e., parallel to the longitudinal axis LA of the plunger 230). The forces F1 and F2 resulting from the pressures P1 and P2 acting upon the plunger 230 are defined by equations (1) and (2) below.

F1=P1*A1 (1)

F2=P2*A2 (2)

Where A1 and A2 are the surface area of the ends of plunger 230 exposed to the pressures P1 and P2, respectively. Also included in the force balance is a frictional force Ff acting in a direction opposing the movement of the plunger 230 (i.e., in a direction parallel to the longitudinal axis LA of the plunger 230). Under steady-state conditions (i.e., when the plunger 230 is moving at a constant velocity V, the relationship between the forces F1, F2 and Ff is given by equation (3):

F1=F2+Ff (3)

Substituting equations (1) and (2) results in equation (4), which defines the relationship between the pressure P2 of the therapeutic fluid and the pressure P1 of the working fluid:

P2=(A1/A2)*P1−Ff/A2. (4)

As illustrated by equation (4), the delivery pressure P2 of the therapeutic fluid in the second portion 216 can be controlled by controlling the pressure P1 of the working fluid in the first portion 214, by adjusting the area ratio (A1/A2) of the plunger 230 and/or by controlling the frictional force Ff. The frictional force Ff can be minimized, for example, by including a low friction sealing member between the plunger and the side wall of the reservoir, as will be discussed in more detail herein.

Although FIG. 6 and the equations above include only the pressure forces F1, F2 and the frictional force Ff, in some embodiments, additional forces act on the plunger. For example, as discussed in more detail herein, in some embodiments, the delivery device includes a biasing member configured to produce a force on one or both sides of the plunger. Moreover, although equation (4) presents the relationship between the pressures P1 and P2 under steady-state conditions, a similar relationship exists between the pressures P1 and P2, the velocity V of the plunger and the flow rate of the therapeutic fluid under transient conditions.

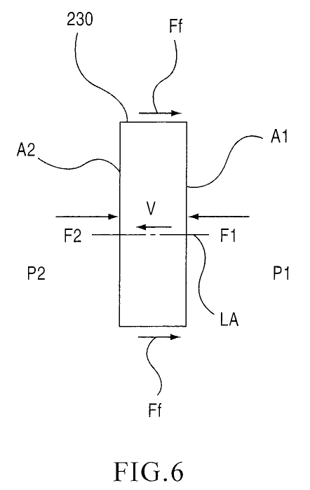

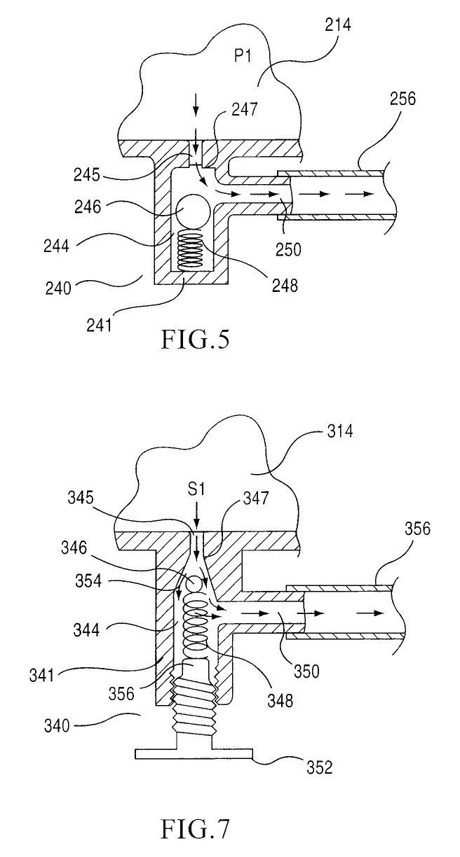

The pressure P1 of the working fluid is controlled by the valve 240. As illustrated in FIGS. 4 and 5 and described herein, the valve 240 controls the pressure P1 of the working fluid to a predetermined maximum value by permitting a portion of the working fluid to be discharged from the first portion 214 of the reservoir 210 when the pressure P1 exceeds the predetermined maximum value. The valve 240 includes a housing 241 that defines a valve containment area 244, a sealing portion 247 and a bypass flow area 250. The valve containment area 244 contains a spring 248 and a check ball 246. In use, when the pressure P1 of the working fluid in the first portion 214 of the reservoir 210 is below the predetermined maximum value, the force exerted by the spring 248 on the check ball 246 exceeds the force exerted by the pressure P1 on the check ball 246. As such, the check ball 246 is disposed against the sealing portion 247 thereby forming a fluid-tight seal between the first portion of the reservoir 214 and the valve containment area 244 (see FIG. 4). When the pressure P1 of the working fluid exceeds the predetermined maximum value, the force exerted by the pressure P1 on the check ball 246 exceeds the force produced by the spring 248, thereby causing the check ball 246 to be displaced away from the sealing portion 247, placing the valve 240 into the bypass configuration (see FIG. 5). In the bypass configuration, the first portion 214 of the reservoir 210 is in fluid communication with the valve containment area 244 via orifice 245, thereby allowing a portion of the working fluid to flow from the first portion 214 of the reservoir into the bypass flow area 250 and through the drain tube 256.

In the illustrated embodiment, a user can ensure that the pressure P1 of the working fluid is at or near the maximum pressure value by introducing the working fluid into the first portion 214 of the reservoir 210 at a rate such that valve 240 remains in the bypass configuration. Put another way, a condition in which the pressure of the working fluid is too low can be avoided by introducing the working fluid into the first portion 214 at a rate such that the flow indicator 258 produces an indication that fluid is flowing through the drain tube 256.

In some embodiments, the housing 241 of the valve 240 and the reservoir 210 are monolithically formed. In other embodiments, the valve can be a separate component that is coupled to the reservoir. The valve can be, for example, threadably coupled to the reservoir, welded to the reservoir, coupled to the reservoir via an adhesive or coupled to the reservoir by any other suitable method.

The valve set point (i.e., the point at which the valve 240 transitions from the closed configuration to the bypass configuration) is a function of, among other things, the size of the orifice 245, the mass and size of the check ball 246 and the free length and spring constant of the spring 248. As illustrated in FIGS. 4 and 5, the valve set point cannot be adjusted unless the hardware is changed and/or modified. In some embodiments, however, the valve can include a mechanism for adjusting the valve set point, as will be discussed herein.

The reservoir 210 can be constructed from any material suitable for containing therapeutic fluids. Such materials can include, for example, a variety of plastics, glass, stainless steel and/or composite materials. In some embodiments, the reservoir 210 can be monolithically formed from a single material. In other embodiments, the reservoir 210 can be formed from a variety of materials such that the material properties vary spatially. In this manner, the material properties of the first portion 214 of the reservoir 210 can be different from those of the second portion 216 of the reservoir 210. In yet other embodiments, the inner surface of the second portion 216 of the reservoir 210 can be coated with a material known to enhance the survival of living cells, such as, for example, proteins, sodium alginate, and/or various polymers.

Although described as having a cylindrical shape with a constant inner diameter D, the reservoir 210 can have any shape. For example, in some embodiments, the reservoir 210 can have an elliptical cross-sectional shape, a rectangular cross-sectional shape, a conical cross-sectional shape, and the like. Moreover, the inner dimensions, such as the diameter D, of the reservoir need not be constant, but can vary along the length of the reservoir. In this manner, as the plunger moves, the change in the volume of the first portion 214 is not equal to the change in the volume of the second portion 216, as will be discussed in more detail herein.

The plunger 230 can be constructed from any material suitable for movably dividing the reservoir 210 into the first portion 214 and the second portion 216 while preventing fluid communication between the two portions of the reservoir 210. Moreover, because at least a portion of the plunger 230 is in contact with the therapeutic fluid contained within the second portion 216 of the reservoir 210, the plunger 230 can be constructed from a material that is compatible with the therapeutic fluid. Such materials can include, for example, a variety of plastics, stainless steel and/or composite materials. In some embodiments, the plunger 230 can be monolithically formed from a single material. In other embodiments, the plunger 230 can be constructed from distinct components. For example, in some embodiments, the plunger can include a plunger and a sealing member, configured to create a substantially fluid-tight seal between the first portion 214 of the reservoir and the second portion 216 of the reservoir 210. Such sealing members can include, for example, o-rings or sealing rings constructed from polytetraflouroethylene or any other suitable polymer. In other embodiments, one or more surfaces of the plunger can include a coating configured to enhance the survival of living cells. In yet other embodiments, one or more surfaces of the plunger can include a coating configured to reduce the friction between the plunger and the inner surfaces of the reservoir 210.

FIG. 7 is cross-sectional view of a valve 340 according to an embodiment of the invention. The valve 340 is similar to the valve 240 described above in that it includes a housing 341 that defines a valve containment area 344, a sealing portion 347 and a bypass flow area 350. The valve containment area 344 contains a spring 348 and a check ball 346. As described above, when the pressure P1 of the working fluid within the first portion 314 of the reservoir is below the valve set point, the force exerted by the spring 348 on the check ball 346 is sufficient to hold the check ball 346 against the sealing portion 347 thereby forming a seal preventing fluid communication between the first portion 314 of the reservoir and the valve containment area 344 (not shown in FIG. 7).

When the pressure P1 of the working fluid S1 exceeds the valve set point, the force exerted by the pressure of the working fluid on the check ball 346 exceeds the force exerted by the spring 348, thereby causing the check ball 346 to be displaced away from the sealing portion 347, as shown in FIG. 7. When in the bypass configuration, the first portion 314 of the reservoir is in fluid communication with the valve containment area 344 via orifice 345, as described above. The pressure control device 340 also includes a pressure adjustment knob 352 that is threadably engaged with the housing 341. The pressure adjustment knob 352 includes a spring seat portion 356, against which one end of the spring 348 is disposed. The length of the spring 348, and therefore the force produced by the spring 348 on the check ball 346, can be adjusted by rotating the adjustment knob 352 within the housing 341.

The valve containment area 344 of the pressure control device 340 includes a tapered portion 354 adjacent the sealing portion 347. The tapered portion 354 defines a bypass flow area of the pressure control device 340 that increases as the check ball 346 moves away from the sealing portion 347. By increasing the bypass flow area, the pressure control device 340 allows a greater amount of working fluid to be discharged from the first portion 314 as the pressure P1 increases above the maximum pressure set point, thereby allowing for more precise control of the pressure P1. Although the tapered portion 354 is illustrated as having a linear cross-sectional shape, in some embodiments, the tapered portion can be of any shape suitable for providing the optimal pressure control characteristics. For example, in some embodiments, the tapered portion 354 can be curved in shape. In other embodiments, the tapered portion can include a discontinuous portion.

Although the delivery device 300 is shown as including one valve 340, in some embodiments, the delivery device can include two or more pressure control devices. For example, in some embodiments, the delivery device includes a first pressure control device having a first pressure set point and a second pressure control device having a second pressure set point greater than the first pressure set point. In this manner, the second pressure control device provides a secondary bypass flow area to allow a greater amount of the working fluid to be removed from the first portion of the reservoir when the pressure exceeds the second pressure set point. Moreover, in some embodiments, both the first and the second pressure control devices can each include a drain tube, each having a flow indicator as discussed above. In use, a user can ensure that the pressure of the working fluid in the first portion of the reservoir is between the first pressure set point and the second pressure set point by introducing the working fluid into the first portion at a rate such that the flow indicator included in the drain tube of the first pressure control device produces an indication that fluid is flowing through the drain tube while the flow indicator included in the drain tube of the second pressure control device produces an indication that no fluid is flowing through the drain tube.

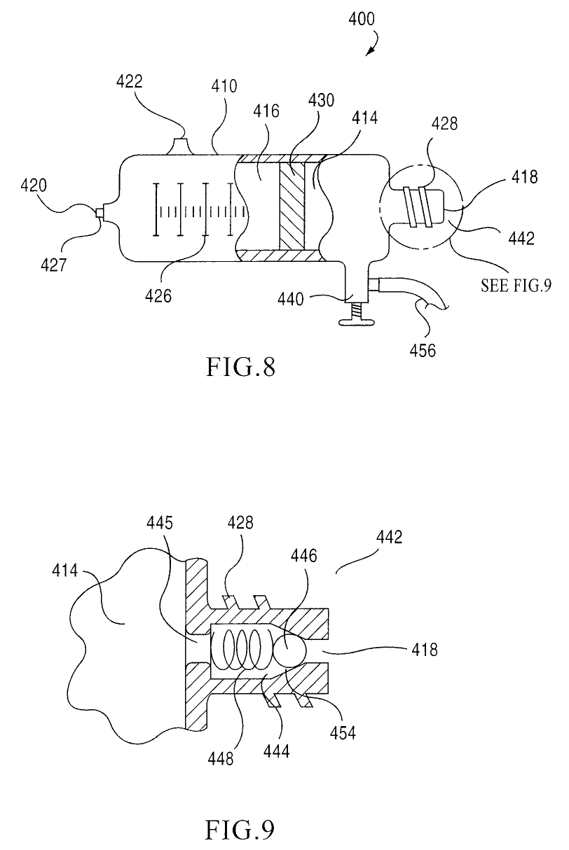

FIGS. 8 and 9 illustrate a delivery device 400 according to an embodiment of the invention that includes a reservoir 410 configured to contain a fluid and a plunger 430 disposed within the reservoir 410. The plunger 430 is disposed within the reservoir 410 such that the reservoir 410 is divided into a first portion 414 and a second portion 416 that is fluidically isolated from the first portion 414. As described above, the reservoir 410 is configured such that as the plunger 430 moves, the change in the volume of the first portion 414 is equal to the change in the volume of the second portion 416. The reservoir 410 includes a graduated set of markings 426 configured to allow a user to determine a position of the plunger 430. In some embodiments, the graduated set of markings 426 corresponds a linear distance. In other embodiments, the graduated set of markings 426 corresponds to the volume of the second portion 416 of the reservoir 410. In some embodiments, to accommodate tracking of the plunger 430, the portion of the reservoir 410 that includes the graduated set of markings 426 can be transparent. In other embodiments, the plunger 430 can include an indicator, such as a red marking, that can be easily seen through the reservoir 410.

The reservoir 410 includes a first port 418 in fluid communication with the first portion 414 of the reservoir 410. As illustrated, the first port 418 includes external threads 428 to facilitate the attachment of a fluid supply device (not shown), such as a syringe. The reservoir 410 also includes a second port 420 and third port 422, each being in fluid communication with the second portion 416 of the reservoir 410. As described above, the second port 420 can be connected to a device configured to deliver a therapeutic fluid to a target location, while the third port 422 can be used, for example, as a fill port. The delivery device 400 also includes a first pressure control valve 440 of the type described above.

As illustrated, the first port 418 includes a second pressure control valve 442 configured to ensure that the supply pressure of the working fluid is above a minimum pressure set point. The pressure control device 442 includes a housing 441 that defines a valve containment area 444, a sealing portion 447 and an orifice 445. The valve containment area 444 contains a spring 448 and a check ball 446. In use, when the pressure of the working fluid being conveyed by a fluid supply source (not shown) is below the minimum pressure set point, the force exerted by the spring 448 on the check ball 446 exceeds the force exerted by the supply pressure of the working fluid on the check ball 446. As such, the check ball 446 remains disposed against the sealing portion 447 thereby preventing the flow of working fluid from the fluid supply source into the first portion 414.

When the pressure the working fluid being conveyed by a fluid supply device exceeds the minimum pressure set point, the force exerted by the pressure of the working fluid on the check ball 446 exceeds the force produced by the spring 448, causing the check ball 446 to be displaced away from the sealing portion 447, thereby allowing the flow of working fluid into the first portion 414 via the port 418. By allowing the working fluid to flow into the first portion 414 only when the supply pressure exceeds a minimum value, the second pressure control device 442 ensures that the pressure within the first portion 414 will be above the minimum pressure set point. In this manner, the delivery device 400 is configured to ensure that the pressure of the working fluid will be controlled to a value greater than that of the minimum pressure set point of the second pressure control valve 442 and less than that of the maximum pressure set point of the first pressure control valve 440.

In some embodiments, the second pressure control valve 442 and the first port 418 are monolithically formed with the reservoir 410. In yet other embodiments, the second pressure control valve and the first port can be separate components that are coupled to the reservoir. The second pressure control valve and the first port can be, for example, threadably coupled to the reservoir, welded to the reservoir, coupled to the reservoir via an adhesive or coupled to the reservoir by any other suitable method.

As described above, the pressure set point of the second pressure control valve 442 is a function of, among other things, the mass and size of the check ball 446 and the properties of the spring 448. In some embodiments, the minimum pressure set point of the second pressure control valve is not readily adjustable without changing the hardware, such as the spring 448 and/or check ball 446. In other embodiments, the second pressure control valve can include a mechanism to adjust the minimum pressure set point. In some embodiments, the minimum pressure set point of the second pressure control valve 442 is less than the maximum pressure set point of the first pressure control valve 440. In this manner, the two pressure control valves 440, 442 are configured to cooperatively maintain the pressure of the working fluid within the range defined by the two set points. In other embodiments, the minimum pressure set point of the second pressure control valve 442 is equal to or greater than the maximum pressure set point of the first pressure control valve 440. In this manner, the two pressure control valves 440, 442 are configured to cooperatively maintain the pressure of the working fluid at substantially a constant value (i.e., the maximum pressure set point) at all times.

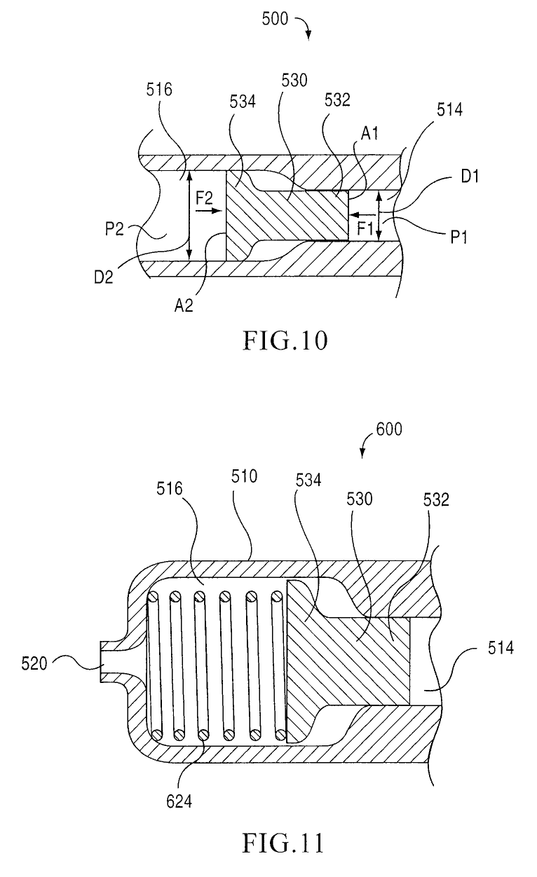

FIG. 10 is a cross-sectional view of portion of a delivery device 500 according to an embodiment of the invention that includes a reservoir 510 and a plunger 530 disposed within the reservoir 510 such that the reservoir 510 is divided into a first portion 514 and a second portion 516. As illustrated, the first portion 514 has an inner diameter D1 and the second portion 516 has an inner diameter D2 that is greater than the inner diameter D1. The plunger 530 contains a first portion 532 disposed within the first portion 514 of the reservoir 510 and a second portion 534 disposed within the second portion 516 of the reservoir. The end of the first portion 532 of the plunger 530 has a cross-sectional area A1 measured along a plane normal to the longitudinal axis of the plunger 530. The cross-sectional area A1 is directly proportion to the inner diameter D1 squared. Similarly, the end of the second portion 534 of the plunger 530 has a cross-sectional area A2 directly proportional to the inner diameter D2 squared.

In contrast to the embodiments described above, because the inner diameter D1 of the first portion 514 and the inner diameter D2 of the second portion 516 are unequal, as the plunger 530 moves, the change in the volume of the first portion 514 is not equal to the change in the volume of the second portion 516. Rather, the relationship between the change in volume of the first portion 514 and the change in volume of the second portion 516 is proportional to the ratio of the cross-sectional areas A1 and A2 (or the ratio of the diameters D1 and D2, squared). Put another way, when the delivery device 500 is in use, the flow rate of the therapeutic fluid exiting the second portion 516 (i.e., the change in volume of the second portion 516) is related to the flow rate of the working fluid entering the first portion 514 (i.e., the change in volume of the first portion 514) by Equation (5), below.

Q2=(A2/A1)*Q1 (5)

Where Q1 and Q2 are the flow rates of the working fluid and the therapeutic fluid, respectively. By modifying the area ratio, the delivery device 500 can be configured to provide more accurate control of the flow rate of the therapeutic fluid exiting the second portion.

Similarly, as previously presented in Equation 4 (reproduced below), under steady-state conditions, the relationship between the pressure P2 of the therapeutic fluid and the pressure P1 of the working fluid is also a function of the ratio of the cross-sectional areas A1 and A2. In this manner, by configuring the reservoir 510 such that D2 is greater than D1, the pressure P2 in the second portion 516 of the reservoir 510 will be less than the pressure P1 in the first portion 514 of the reservoir 510, which enables more accurate control of the pressure P2.

P2=(A1/A2)*P1−Ff/A2 (4)

Although the reservoir 510 is described as having a cylindrical shape with inner diameters D1 and D2, the reservoir 510 can have any shape. For example, in some embodiments, the reservoir 510 can have an elliptical cross-sectional shape, a rectangular cross-sectional shape, a conical cross-sectional shape, and the like. Moreover, the inner diameters D1 and D2 need not be constant, but can vary along the length of the reservoir. In this manner, the relationship between the flow rates of the working fluid and therapeutic fluid can change as a function of the position of the plunger 530.

FIG. 11 is a cross-sectional view of portion of a delivery device 600 according to an embodiment of the invention that includes a reservoir 510 and a plunger 530 disposed within the reservoir 510 such that the reservoir 510 is divided into a first portion 514 and a second portion 516. As described above, the first portion 514 has an inner diameter D1 and the second portion 516 has an inner diameter D2 that is greater than the inner diameter D1. The plunger 530 contains a first portion 532 disposed within the first portion 514 of the reservoir 510 and a second portion 534 disposed within the second portion 516 of the reservoir. The delivery device 600 differs from the delivery device 500 described above in that delivery device 600 includes a biasing member 624, such as a spring, disposed within the second portion 516 of the reservoir 510 that produces a force against the second portion 534 of the plunger 530. The addition of the biasing member 624 changes the relationship between the pressure P2 of the therapeutic fluid and the pressure P1 of the working fluid, as indicated in Equation (6).

P2=(A1/A2)*P1−Ff/A2−(X*Ks)/A2 (6)

Where Ks is the spring constant of the biasing member 624 and X is the linear displacement of the spring, which corresponds to the position of the plunger 530. In this manner, the pressure of the therapeutic fluid in the second portion 516 is reduced as the volume of the second portion 516 decreases (i.e., as the plunger 530 moves). Such an arrangement allows the delivery pressure of the therapeutic fluid to be controlled as a function of the amount of therapeutic fluid delivered.

Although illustrated and described as a spring having a constant spring constant, the biasing member need not be so limited. In some embodiments, for example, the biasing member can be a spring having a variable spring constant. Moreover, in some embodiments, a biasing member can be included to produce a force on the first portion 532 of the plunger 530. In yet other embodiments, the delivery device includes multiple biasing members, configured to produce a force on each of the first portion 532 and the second portion 534 of the plunger 530.

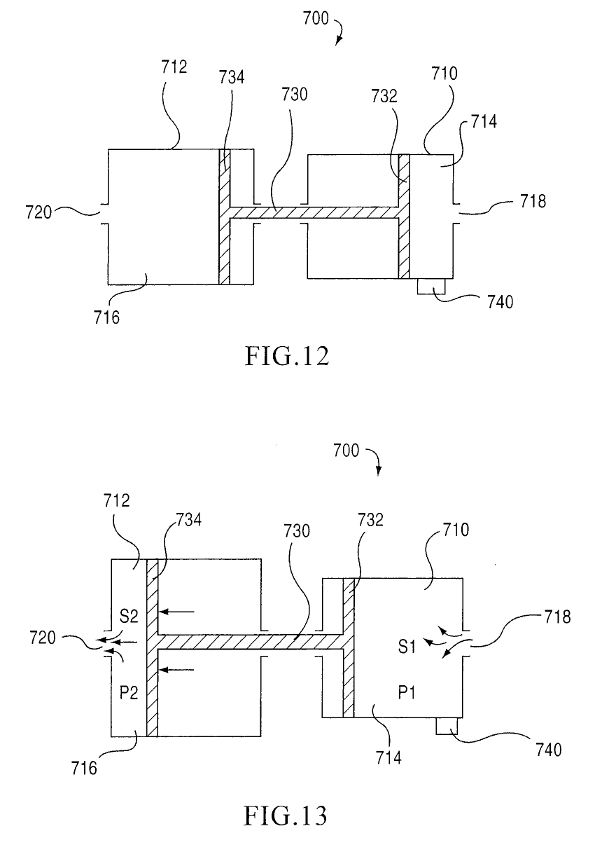

FIGS. 12 and 13 are schematic illustrations of a delivery device 700 according to an embodiment of the invention in a first position and a second position, respectively. The delivery device 700 includes a first reservoir 710, a second reservoir 712, a plunger 730 and a pressure control device 740. The first reservoir 710 includes a fluid containment portion 714 configured to contain a fluid S1, such as, for example a working fluid. Similarly, the second reservoir 712 includes a fluid containment portion 716 configured to contain a fluid S2, such as, for example, a therapeutic fluid containing living cells. The plunger 730 includes a first portion 732 disposed within the first reservoir 710 and a second portion 734 disposed within the second reservoir 712. As will be described in more detail herein, the first reservoir 710 and the second reservoir 712 are fixed relative to each other. In this manner, movement of the first portion 732 of the plunger 730 within the first reservoir 710 corresponds to movement of the second portion 734 of the plunger 730 within the second reservoir 712. In some embodiments, for example, the first reservoir 710 can be coupled to the second reservoir 712 via any suitable manner, such as, for example, by a threaded coupling, an interference fit between a portion of the first reservoir 710 and the second reservoir 712, or the like.

The first portion 732 of the plunger 730 is configured to create a substantially fluid-tight seal between the fluid containment portion 714 and the remaining portions of the first reservoir 710. In this manner, the fluid S1 introduced into to the fluid containment portion 714, will not leak past the first portion 732 of the plunger 730, but instead will cause corresponding movement of the plunger 730 within the second reservoir 712. Similarly, the second portion 734 of the plunger 730 is configured to create a substantially fluid-tight seal between the fluid containment portion 716 and the remaining portions of the second reservoir 712.

The first reservoir 710 includes a first port 718 in fluid communication with the fluid containment portion 714. As discussed above, the first port 718 can be, for example, an inlet port configured to be connected to a fluid supply source, such as a syringe. Similarly, the second reservoir 712 includes a second port 720 in fluid communication with the fluid containment portion 716.

The pressure control device 740 is in fluid communication with the fluid containment portion 714 of the first reservoir 710. As described above, the pressure control device 740 can be any suitable device, such as a check valve, configured to control a pressure P1 of a first fluid S1 within the fluid containment portion 714 of the first reservoir 710.

In use, the fluid containment portion 716 of the second reservoir 712 is filled with a predetermined amount of the fluid S2. Once filled with the desired amount of the fluid S2, the second port 720 can then be placed in fluid communication with any suitable device (not shown), such as a tube, a catheter, and the like, configured to deliver the fluid S2 into a target location. The fluid S2 can be conveyed from the fluid containment portion 716 of the second reservoir 710, by introducing a fluid S1 into the fluid containment portion 714 of the first reservoir 710. In some embodiments, the fluid S1 can be introduced into the fluid containment portion 714 by a syringe (not shown) in fluid communication with the first port 718. The introduction of the fluid S1 into the fluid containment portion 714 of the first reservoir 710 causes the movable member 730 to move, as shown in FIG. 13, such that the volume of the fluid containment portion 714 of the first reservoir 710 increases and the volume of the fluid containment portion 716 of the second reservoir 712 decreases, thereby causing the fluid S2 to be expelled from the from the second reservoir 712.

As previously described, the pressure P2 and/or rate at which the fluid S2 is delivered from the fluid containment portion 716 of the second reservoir 712 is dependent on the pressure P1 exerted by the fluid S1 on the movable member 730. The pressure P1 of the fluid S1 within the fluid containment portion 114 of the first reservoir 710 is controlled by the pressure control device 740, as described above.

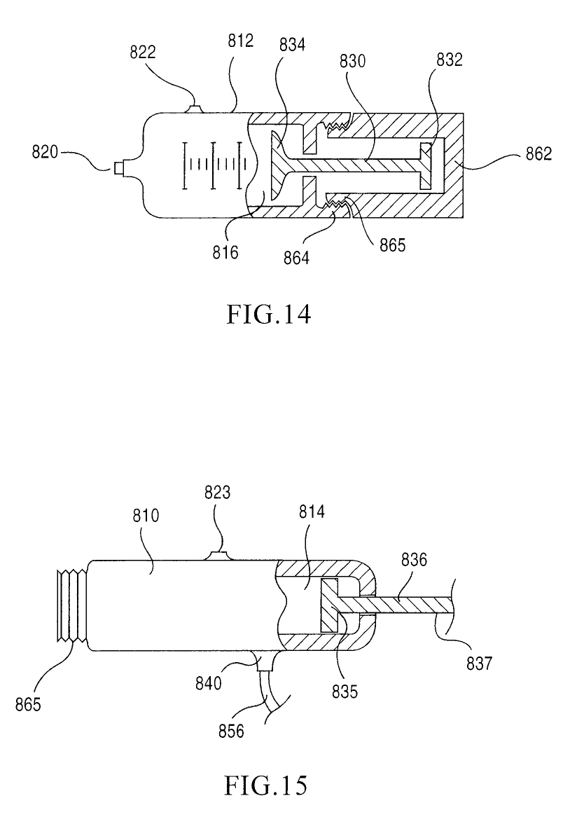

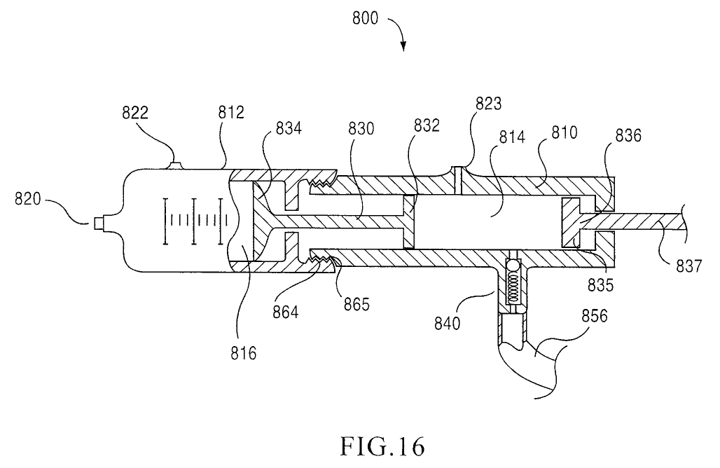

FIGS. 14-16 illustrate a delivery device 800 according to an embodiment of the invention in which a first reservoir 810 can be threadably coupled to a second reservoir 812. FIGS. 14 and 15 illustrate the second reservoir 812 and the first reservoir 810, respectively, in a first configuration, in which they are decoupled from each other. FIG. 16 illustrates the delivery device 800 in a second configuration, in which the first reservoir 810 and the second reservoir 812 are coupled together. Such an arrangement allows the second reservoir 812 to be filled with a therapeutic fluid and stored in a controlled environment apart from the first reservoir 810. For example, in some embodiments, the second reservoir 812 can be filled with a precise amount of living cells and stored at a central laboratory until needed. Such an arrangement also allows for the first reservoir 810 to be reused for subsequent procedures.

The illustrated delivery device 800 includes a first reservoir 810, a second reservoir 812, a first plunger 830, a second plunger 836 and a valve 840. The first reservoir 810 includes a fluid containment portion 814 configured to contain a fluid, such as, for example, a saline solution. Similarly, the second reservoir 812 includes a fluid containment portion 816 configured to contain a fluid, such as, for example, a therapeutic fluid containing living cells.

The first plunger 830 includes a first portion 832 and a second portion 834. The first portion 832 is configured to be disposed within the first reservoir 810 such that the first portion 832 creates a substantially fluid-tight seal between the fluid containment portion 814 and the remaining portions of the first reservoir 810, as shown in FIG. 16. Note that when the delivery device 800 is in the first configuration (i.e., the disassembled configuration, as illustrated in FIGS. 14 and 15), the first portion 832 of the first plunger 830 is disposed within a protective cap 862. The protective cap 862 is removably coupled to the second reservoir 812 by mating threads 864, 865 and prevents the first plunger 830 from being moved within the second reservoir 812 prior to the intended use of the delivery device 800. The protective cap 862 also prevents the first portion 832 of the first plunger 830 from being damaged prior to use. The second portion 834 of the first plunger 830 is disposed within the second reservoir 812 and is configured to create a substantially fluid-tight seal between the fluid containment portion 816 and the remaining portions of the second reservoir 812.

The second plunger 836 contains a first portion 835 and a second portion 837. The first portion 835 of the second plunger 836 is disposed within the first reservoir 810 such that it creates a substantially fluid-tight seal between the fluid containment portion 814 and the remaining portions of the first reservoir 810, as shown in FIGS. 15 and 16. The second portion 837 of the second plunger 836 is disposed outside of the first reservoir 810 and can be configured to allow a user to move the second plunger 836. In some embodiments, for example, the second portion 837 includes a handle, such as the type found on a syringe.

The first reservoir 810 includes a port 823 in fluid communication with the fluid containment portion 814. As described in more detail herein, a working fluid is supplied to the fluid containment portion 814 of the first reservoir 810 via the port 823 when the first reservoir 810 is coupled to the second reservoir 812. Similarly, the second reservoir 812 includes a first port 820 and a second port 822, each in fluid communication with the fluid containment potion 816 of the second reservoir 812. The first port 820 of the second reservoir 812 can be an outlet port configured to be removably connected to a catheter tube or other device for transporting the therapeutic fluid from the delivery device 800 to the targeted area in the patient's body. The second port 822 of the second reservoir 812 can, for example, be a fill port, such as a quick-connect fitting, configured to be removably coupled to a supply of therapeutic fluid. In use, as the therapeutic fluid is being introduced into the second reservoir 812 via the second port 822, the first port 820 can be used to bleed air trapped in the fluid containment portion 814 of the second reservoir 812. The second reservoir 812, however, need not have a second port 822. In such an arrangement, the second reservoir 812 can be filled via the first port 820.

As illustrated in FIG. 16, the valve 840 is in fluid communication with the fluid containment portion 814 of the first reservoir 810. The valve 840 can be any suitable control device, such as those discussed above, configured to control a pressure of a working fluid within the fluid containment portion 814 of the first reservoir 810. In the illustrated embodiment, a drain tube 856 is coupled to the valve 840.

In use, fluid containment portion 816 of the second reservoir 812 can be filled with a predetermined amount of a therapeutic fluid, via port 822 and/or port 820. Once the fluid containment portion 816 is filled with the desired volume of therapeutic fluid and the first plunger 830 is properly positioned, the protective cap 862 can then be placed over the end of the second reservoir 812. Although shown as a threaded connection, the protective cap 862 can be secured to the second reservoir 812 by any suitable means, such as, for example, an interference fit. Upon being filled, the second reservoir 812 can be stored in a controlled environment until needed.

To deliver the therapeutic fluid from the second reservoir 812 to the target location, the protective cap 862 is removed and the first portion 832 of the first plunger 830 is disposed within the first reservoir, as shown in FIG. 16. During this operation, the ports 820 and 822 are closed to prevent any leakage of the therapeutic fluid from the second reservoir 812 as the first plunger 830 is being positioned within the first reservoir 810. The second reservoir 812 is then coupled to the first reservoir 810. Although shown as a threaded connection, the first reservoir 810 and the second reservoir 812 can be coupled in any manner that prevents the second reservoir 812 from moving in an axial direction relative to the first reservoir 810. In some embodiments, for example, the second reservoir 812 can be coupled to the first reservoir 810 by an interference fit. In other embodiments, the second reservoir 812 can be coupled to the first reservoir 810 in a manner that allows the second reservoir 812 to rotate relative to the first reservoir 810. Moreover, although shown as being removably connected, in some embodiments, the second reservoir 812 can be coupled to the first reservoir 810 in a manner that prevents the second reservoir 812 from being removed from the first reservoir 810.

The fluid containment portion 814 of the first reservoir 810 is then filled with a working fluid via port 823. In some embodiments, the fluid containment portion 814 of the first reservoir 810 is filled by connecting port 823 to a fluid supply source (not shown) and displacing the second plunger 836 such that the working fluid is drawn into the fluid containment portion 814. To prevent movement of the first plunger 830 and/or any leakage of the therapeutic fluid from the second reservoir 812, the ports 820 and 822 are closed while the first reservoir 810 is being filled. Upon being filled, the port 823 is closed. In this manner, the delivery device 800 includes a hydraulic link between the first end 835 of the second plunger 836 and the first end 832 of the first plunger 830.

The fluid containment portion 816 of the second reservoir 812 is then connected to a catheter tube (not shown) via port 820. The therapeutic fluid within the second reservoir 812 can be delivered to the target location by pushing the second portion 837 of the second plunger 836. As the second plunger 836 moves axially towards the first plunger 830, the pressure of the working fluid in the fluid containment portion 814 of the first reservoir 810 increases. The increased pressure acts on the first end 832 of the first plunger 830, causing it to move, thereby expelling the therapeutic fluid out of the fluid containment portion 816 via port 820. As described above, the valve 840 limits the pressure of the working fluid in the fluid containment portion 814 by allowing a portion of the working fluid to be discharged from the fluid containment portion 814. In this manner, the pressure and/or flow rate at which the therapeutic fluid is delivered can be controlled.

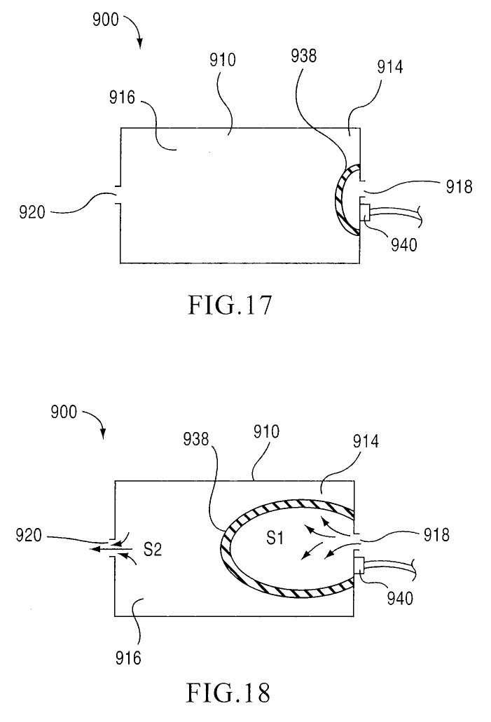

FIGS. 17 and 18 are schematic illustrations of a delivery device 900 according to an embodiment of the invention in a first configuration and a second configuration, respectively. The illustrated delivery device 900 includes a reservoir 910 configured to contain a fluid and an expandable member 938 disposed within the reservoir 910. The expandable member 938, which can be, for example, an elastomeric material, is disposed within the reservoir 910 such that the reservoir 910 is divided into a first portion 914 and a second portion 916 that is fluidically isolated from the first portion 914. The reservoir includes a first port 918 in fluid communication with the first portion 914 and a second port 920 in fluid communication with the second portion 916. As discussed above, the first port 918 can be an inlet port configured to be connected to a fluid supply source, such as a syringe (not shown). The second port 920 can be, for example, be a quick connect fitting configured to allow the second port 920 to be removably connected to a catheter tube for receiving a fluid from the second portion 916 of the reservoir 910. The illustrated delivery device 900 also includes a pressure control device 940 in fluid communication with the first portion 914 of the reservoir 910. As described above, the pressure control device 940 can be any suitable device, such as a check valve, configured to control a pressure P1 of a first fluid S1 within the first portion 914 of the reservoir 910.

The operation of delivery device 900 is similar to that described above. The operation of delivery device 900 differs in that as a working fluid S1 is introduced into the first portion 914 of the reservoir 910, the expandable member 938 does not move axially, as do the plungers previously described, but rather the expandable member 938 expands thereby increasing the volume of the first portion 914 of the reservoir 910 and decreasing the volume of the second portion 916 of the reservoir. In this manner, the expandable member 938 acts to push the fluid S2 containing a therapeutic fluid out of the second portion 916 of the reservoir 910 via port 920.

The pressure and/or rate at which the fluid S2 is delivered from the second portion 916 of the reservoir 910 is a function of the pressure P1 exerted by the working fluid S1 on the expandable member 938. In addition to those variables described above as influencing the delivery pressure P2 of the fluid S2, the delivery pressure P2 can also be a function of the elastomeric or “spring back” properties of the expandable member 938. For example, in some embodiments, the expandable member 938 can be constructed from a relatively low-compliant material. In such embodiments, the properties of the expandable member 938 exert minimal influence on the delivery pressure P2 of the fluid S2. In other embodiments, the expandable member 938 can be constructed from a relatively high-compliant material. In such embodiments, the properties of the expandable member 938 exert a greater influence on the delivery pressure P2 of the fluid S2.

While various embodiments of the invention have been described above, it should be understood that they have been presented by way of example only, and not limitation. For example, although the reservoirs are shown and described as having a cylindrical shape, in some embodiments a delivery device according to the invention can include a reservoir having any shape. For example, in some embodiments, a reservoir can have an elliptical cross-sectional shape, a rectangular cross-sectional shape, a conical cross-sectional shape, and the like. In other embodiments, the dimensions of the reservoir can vary along the length of the reservoir, thereby resulting in a reservoir having a variable shape.

Similarly, although the movable member is shown and described as being a plunger configured to move in an axial direction to change the volumes of portions of the reservoir, in some embodiments a movable member can move rotationally to accomplish such a volume change. For example, in some embodiments, the movable member can be a vane configured to rotate within the reservoir such that the volumes within the reservoir are changed.

Although specific embodiments are shown and described as having specific mechanisms for controlling the pressure of the working fluid and/or the therapeutic fluid, any of the disclosed pressure control mechanisms can be used in any combination to control the pressure of the working fluid and/or the therapeutic fluid.

1. An apparatus, comprising:
a reservoir having an inlet port and an outlet port;
a rigid movable member disposed within the reservoir such that the reservoir is divided into a first portion and a second portion, the inlet port being in fluid communication with the first portion, the outlet port being in fluid communication with the second portion; and
a pressure control device in fluid communication with the first portion of the reservoir, the pressure control device configured to control a pressure of the fluid within the first portion of the reservoir.

2. The apparatus of claim 1, wherein the pressure control device is a valve configured to limit the pressure of the fluid within the first portion of the reservoir.

3. The apparatus of claim 1, wherein the pressure control device is a check valve configured to limit the pressure of the fluid within the first portion of the reservoir to a selectively adjustable pressure setting.

4. The apparatus of claim 1, further comprising a drain tube coupled to the pressure control device, the drain tube configured to provide an indication that the fluid is passing therethrough.

5. The apparatus of claim 1, wherein the pressure control device is a first pressure control device, the apparatus further comprising a second pressure control device in fluid communication with the first portion of the reservoir, the second pressure control device configured to control the pressure of the fluid within the first portion of the reservoir.

6. The apparatus of claim 1, wherein the pressure control device is a first pressure control device, the apparatus further comprising a second pressure control device disposed adjacent the inlet port, the second pressure control device configured to control the pressure of the fluid within the first portion of the reservoir.

7. The apparatus of claim 1, wherein the movable member has a first portion in fluid communication with the first portion of the reservoir and a second portion in fluid communication with the second portion of the reservoir, the first portion of the movable member having a first area and the second portion of the movable member having a second area, the second area being different from the first area.

8. The apparatus of claim 1, further comprising a biasing member disposed in the second portion of the reservoir, the biasing member configured to engage a portion of the movable member.

9. The apparatus of claim 1, wherein the movable member is configured to create a hermetic seal between the second portion of the reservoir and the first portion of the reservoir.

10. The apparatus of claim 1, wherein the inlet port is a first inlet port, the apparatus further comprising a second inlet port in fluid communication with the second portion.

11. The apparatus of claim 1, wherein the reservoir includes an indicia configured to allow a user to determine a position of the movable member.

12-25. (canceled)

26. A kit, comprising:
a reservoir configured to contain a fluid, the reservoir having an inlet port and an outlet port;
a rigid plunger disposed within the reservoir such that the reservoir is divided into a first portion and a second portion, the inlet port being in fluid communication with the first portion, the outlet port being in fluid communication with the second portion;
a pressure control device in fluid communication with the first portion of the reservoir, the pressure control device configured to control a pressure of a fluid within the first portion of the reservoir; and
a fluid supply member configured to supply the fluid to the first portion of the reservoir.

27. The kit of claim 26, further comprising a drain tube coupled to the pressure control device, the drain tube configured to provide an indication that the fluid is passing therethrough.

28. The kit of claim 26, wherein the fluid supply member is a syringe.

29. The apparatus of claim 26, further comprising a low friction sealing member between the plunger and the reservoir.

30. The apparatus of claim 26, wherein the reservoir is constructed of a rigid material.

31. The apparatus of claim 1, wherein the first portion includes a first fluid and the second portion includes a second fluid different from the first fluid, the second fluid including living cells.

32. The apparatus of claim 1, further comprising a low friction sealing member between the movable member and the reservoir.

33. The apparatus of claim 1, wherein the movable member includes lateral ends in slidable contact with the reservoir.

34. The apparatus of claim 1, wherein at least one surface of the movable member includes a coating configured to enhance the survival of living cells.

35. The apparatus of claim 1, wherein the inlet port includes a single opening in a wall of the first portion, and the outlet port includes a single opening in a wall of the second portion.

36. The apparatus of claim 1, wherein the inlet port is located at a first location on the first portion and the pressure control device is located at a second location on the first portion, the second location different from the first location.

37. The apparatus of claim 1, wherein the reservoir is constructed of a rigid material.
